(19) United States
(12) Patent Application Publication
(10) Pub. No.: US 2010/0107263 A1
(43) Pub. Date: Apr. 29, 2010

(54) C-REACTIVE PROTEIN (CRP) KNOCKOUT MOUSE

(75) Inventors: Steven Kerr, Ridgefield, CT (US); Huiping Jiang, Ridgefield, CT (US); Jeffrey Madwed, Ridgefield, CT (US)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: Boehringer Ingelheim International GMBH & co., KG, Ingelheim (DE)

(21) Appl. No.: 12/523,654

(22) PCT Filed: Jan. 22, 2008
(86) PCT No.: PCT/US08/51665

§ 371 Date: Dec. 4, 2009

Related U.S. Application Data
(60) Provisional application No. 60/885,885, filed on Jan. 20, 2007.

(51) Int. Cl. G01N 33/00; A01K 67/00; C12N 5/00; A01N 1/00; C12N 15/63; C12Q 1/02

(52) U.S. Cl. 800/3; 800/13; 800/18; 800/14; 800/17; 800/15; 435/325; 435/1.1; 435/320.1; 435/29

(57) ABSTRACT

The instant invention relates to a transgenic, non-human animal that carries a mutation in the gene encoding C-reactive protein (CRP). Preferably, the invention relates to an animal comprising a homozygous CRP-deficient mouse and techniques for producing such animals. The invention also relates to organs, tissues, cells, cell lines and sub-cellular fractions derived from such animals. Techniques for generating total or tissue-specific CRP knockout animals are also described. The invention further relates to the use of such knockout animals for the study of the role of CRP proteins in vivo or ex vivo, particularly in relation to its role in inflammatory pathway and in the etiology human diseases.

### CROSS-REFERENCE TO RELATED APPLICATIONS

This application claims the benefit of earlier-filed U.S. Provisional Application Ser. No. 60/858,858, filed Jan. 20, 2007 which is incorporated herein by reference in its entirety.

### BACKGROUND OF THE INVENTION

The instant invention relates to a transgenic, non-human animal that carries a mutation, preferably of germ-line origin, in the gene encoding C-reactive protein (CRP) or a homolog thereof.

The CRP protein or polypeptide as used herein is a member of the pentraxin family of proteins. It should not be confused with C-peptide or Protein C. CRP is a member of a family of calcium-dependent ligand-binding plasma proteins, the other member of which in humans is serum amyloid P component (SAP). The human CRP molecule (Mr 115,135) is composed of five identical nonglycosylated polypeptide subunits (Mr 23,027), each containing 206 amino acid residues. The protomers are non-covalently associated in an annular configuration with cyclic pentameric symmetry. The crystal structure of human CRP demonstrates a pentameric structure and provides insight into the molecular mechanisms by which this highly conserved plasma protein exerts a biological role (Shrive et al., Nat Structural Biol., vol. 3, pp. 346-354, 1996).

The gene encoding CRP has been found to be evolutionarily well-conserved among several animal species. These include, but are not limited to:

(A) Xenopus laevis (African clawed frog) CRP protein: 238 aa (GI:295526)

(B) Mus musculus (house mouse) CRP protein: 225 aa (GI:295904)

(C) Cavia (guinea pigs) CRP protein: 225 aa (GI:300221)

(D) Rattus norvegicus (Norway rat) CRP protein: 230 aa (GI: 203592)

(E) Oryctolagus cuniculus (rabbit) CRP protein: 225 aa (GI:986939)

(F) Sus scrofa (pig) CRP protein: 222 aa (GI: 55742770)

(G) Homo sapiens (human) CRP protein: 224 aa (GI: 30224)

Homologs of CRP gene include, but are not limited to, the hereinbefore described serum amyloid P protein (APCS). Both CRP and APCS belong to pentraxin family of proteins, and comprise a characteristic arrangement of five non-covalently bound subunits.

The human CRP gene is located on chromosome 1q21-q23 spanning approximately 1.9 kb and containing two exons separated by a single intron. The first exon encodes a signal peptide and the first 2 amino acids of the mature protein. This is followed by a 278-nucleotide-long intron that includes a GT repeat sequence. The second exon encodes the remaining 204 amino acids, followed by a stop codon.

CRP is a member of the class of acute phase reactants as its levels rise dramatically during inflammatory processes occurring in the body. This increment is due to a rise in the plasma concentration of IL-6, which is produced by macrophages, endothelial cells and T-cells as well as adipocytes. CRP binds to phosphorylcholine on microbes. It is thought to assist in complement binding to foreign and damaged cells and enhances phagocytosis by macrophages, which express a receptor for CRP. It is also believed to play an important role in innate immunity, as an early defense system against infections.

CRP is also thought to be involved in mounting an inflammatory response through activation of the complement cascade. CRP has been shown to be involved in the innate immune response to infection in humans and has also been implicated in underlying inflammatory and autoimmune diseases. Recently, CRP has been linked to cardiovascular disease. There is epidemiological evidence that suggests baseline CRP levels correlate with increased levels of coronary events such as acute myocardial infarction (Sabatine et al., Circulation 2007; 115; 1528-1536; Ridker et al., Tex Heart Inst J. 2005; 32(3): 384-386).

Assigning a causal role to CRP in cardiovascular disease has been problematic due to the lack of both pharmacological inhibitors of CRP and appropriate rodent models. Although mice over-expressing human CRP have been engineered (Danenberg et al., Circulation. 2003; 108:512), there have been no reports to date of a mouse strain with a loss of CRP function.

### BACKGROUND OF THE INVENTION

The instant invention provides an animal that is deficient in the expression of the endogenous CRP gene, including methods for making such animal, comprising, for example, knockout technology.

The CRP knockout mouse of the instant invention was confirmed to be deficient for both CRP mRNA and CRP protein using routine analytical procedures. With respect to the phenotype, it was found that the immunological phenotype of the homozygous knockout animal was different from the wild-type mouse at least on two levels. Firstly, the homozygous knockout mice of the instant invention showed decreased LPS-stimulated production of TNFα and IL-10 in vivo. The CRP knockout mouse of the instant invention is thus valuable for screening agents which elevate the level of these cytokines. Furthermore, this observed decrease in cytokine production in CRP deficient mice suggests that CRP is more than just a marker of inflammation but acts to modulate the inflammatory response invoked by LPS.

Secondly, studies with the CRP knockout mouse of the instant invention also demonstrated that homozygous knockout animals are characterized by attenuated INFγ and IL-2 production in response to anti-CD3 antibody. This attenuated cytokine production was not observed after mitogenic stimulation with SEB or ConA. This suggests that CRP plays a specific role in T-cell activation, possibly leading to T-cell receptor-induced cytokine production. The observation that CRP is involved with T-cell responses was unexpected. It is possible that CRP mediates these effects by affecting T-cell maturation and/or indirectly effecting T-cell/monocyte interactions. In addition, these effects may be mediated via modulation of T-cell signaling.

CRP's involvement in the humoral immune response was demonstrated by the increase in T-cell independent IgM antibody production induced by immunization with TNP-ficoll. This observation is supported by data from human CRP transgenic mice which over-express human CRP and show a decrease in IgM antibody production after TNP-ficoll immunization. The effects on cytokine production observed in these CRP knockout mice indicate that CRP may indeed modulate the inflammatory response even though stimulated CRP levels are much lower than in humans. These observations suggest that modulation of CRP activity may be therapeutically beneficial for cardiovascular diseases which have an underlying inflammatory component such as atherosclerosis.

The instant invention thus provides for a knockout animal which serves as valuable tool for the study of CRP gene function in vivo. Representative examples of such functions include, but are not limited to, a role of CRP in innate immunity, complement activation, inflammatory response, as well in the etiology of diseases such as autoimmune disorders, cardiovascular diseases, and other inflammatory conditions. Such inflammatory conditions may include, but are not limited to, inflammatory bowel disease (IBD), collagen-induced arthritis (CIA), acute inflammation, asthma, etc.

Preferably, the animal of the instant invention is a mammal. Such include, but is not limited to, the hereinbefore described mouse, guinea pig, rat, rabbit, pig, or goat.

Most preferably, the instant invention relates to a non-human mammal such as mouse, guinea pig, rat, or rabbit which is deficient in expression of an endogenous CRP gene. The deficiency may include altered expression of at least one of the following proteins:

(A) Mus musculus (house mouse) CRP protein: 225 aa (GI:295904)

(B) Cavia (guinea pigs) CRP protein: 225 aa (GI:300221)

(C) Rattus norvegicus (Norway rat) CRP protein: 230 aa (GI: 203592)

(D) Oryctolagus cuniculus (rabbit) CRP protein: 225 aa (GI:986939)

As used herein the terms “disruption,” “functional inactivation,” “alteration” and “defect” connote a partial or complete reduction in the expression and/or function of the CRP polypeptide encoded by the endogenous gene of a single type of cell, selected cells or all of the cells of a CRP knockout animal. Thus, according to the instant invention the expression or function of the CRP gene product can be completely or partially disrupted or reduced (e.g., by 50%, 75%, 80%, 90%, 95% or more, e.g., 100%) in a selected group of cells (e.g., a tissue or organ) or in the entire animal. As used herein the term “a functionally disrupted CRP gene” includes a modified CRP gene that either fails to express any polypeptide product or that expresses a truncated protein having less than the entire amino acid polypeptide chain of a wild-type protein and is non-functional (partially or completely non-functional).

The term “knockout animal” refers to an animal comprising a partial or complete reduction of the expression of at least a portion of a polypeptide encoded by an endogenous gene (such as CRP) in a single cell, selected cells, or all of the cells of said animal. The animal may be “heterozygous,” wherein one allele of the endogenous gene has been disrupted. Alternatively, the animal may be “homozygous” wherein both alleles of the endogenous gene have been disrupted.

Disruption of the CRP gene can be accomplished by a variety of methods known to those of skill in the art. For example, gene targeting using homologous recombination, mutagenesis (e.g., point mutation), RNA interference and antisense technology can be used to disrupt a CRP gene.

More specifically, the invention provides a knockout mammal, e.g. mouse, whose genome comprises either a homozygous or heterozygous disruption of its CRP gene. A knockout mammal whose genome comprises a homozygous disruption is characterized by somatic and germ cells that contain two nonfunctional (disrupted) alleles of the CRP gene, while a knockout mammal whose genome comprises a heterologous disruption is characterized by somatic and germ cells that contain one wild-type allele and one nonfunctional allele of the CRP gene.

The type of gene disruption can be global (i.e., wherein every cell of an animal is deficient in the gene) or tissue-specific (i.e., wherein disruption of the gene is limited to one or more tissues). In addition, disruption can be achieved at specific time points (i.e., time-specific knockout) using art known techniques.

Preferably, the animals of the instant invention are global knockouts that are deficient in the endogenous CRP gene.

Particularly preferable are animals that comprise homozygous disruption of the CRP gene. Such animals are characterized by the genotype CRP^{-/-}. As hereinbefore described, the CRP^{-/-} genotype may be manifested globally or in a tissue-specific manner using art known knockout techniques.

As used herein, the term “genotype” refers to the genetic makeup of an animal. A particular genotype refers to one or more specific genes, e.g., CRP. More specifically the term genotype refers to the status of the animal's CRP alleles, which can either be intact and functional (e.g., wild-type or ^{+/+}); or disrupted (e.g., knockout) in a manner that confers either a heterozygous (e.g., ^{+/-}), or homozygous (e.g., ^{-/-}) knockout genotype.

Most preferably, the animal of the instant invention is a mouse which comprises a germline disruption of the gene encoding mouse C-reactive protein (mCRP). The mice may be heterozygous (characterized by the genotype CRP^{+/-}) or homozygous (characterized by the genotype CRP^{-/-}) for the disrupted CRP allele.

In one special embodiment, the instant invention relates to a CRP^{-/-} mouse containing a germline disruption of a single allele encoding mouse CRP.

In another embodiment, the instant invention relates to a CRP^{-/-} mouse containing a germline disruption of both alleles encoding mouse CRP.

The CRP gene can comprise one or more exons. As is understood in the art, an exon is any region of DNA within a gene that is transcribed to the RNA molecule, rather than being spliced. By the way of a representative example, the organization of exons in mouse CRP is shown in FIG. 1. As disclosed therein, the CRP gene in mouse comprises two exons. Thus in the instant invention, there is provided a knockout animal comprising disruption of one or more exon regions. The disruption may comprise complete or partial deletion of exon 1, exon 2 or both exons 1 and 2.

Preferably, the transgenic knockout animal of the instant invention comprises a complete deletion of a major exon which encodes a portion of mature CRP protein. In mice, a major exon comprises exon 2 of the CRP gene.

The transgenic animals of the instant invention are characterized by at least one differential phenotype compared to wild-type animals. Such differential phenotypes may be manifested between wild-type and heterozygous (CRP^{+/-}) knockout animals of the instant invention or between wild-type and homozygous (CRP^{-/-}) knockout animals of the instant invention. In addition, differential phenotypes may be manifested between heterozygous and homozygous knockout animals. Such characteristics or traits may be distinguished at the molecular, biochemical, physiological, pathological and/or behavioral level.

In one embodiment, the knockout mouse of the instant invention comprises an altered phenotype compared to an animal having a wild type CRP gene, wherein said altered phenotype is:
(1) reduced cytokine production after LPS challenge;
(2) reduced T-cell cytokine production after α-CD3 stimulation;
(3) elevated T-cell independent antibody production after immunization with TNP-ficoll; or
(4) any combination of (1), (2) and (3).

In a preferred embodiment, the knockout mouse of the instant invention comprises an altered phenotype compared to an animal having a wild type CRP gene, wherein said altered phenotype is:
(1) reduced TNF-α and IL-10 cytokine production after LPS challenge;
(2) reduced INF-γ and IL-2 production after α-CD3 stimulation;
(3) elevated IgM antibody production; or
(4) any combination of (1), (2) and (3).

The CRP deficient mice of the present invention mice were put through a battery of inflammatory and immunological tests to identify a potential functional role of CRP deficiency. In these studies, compared to wild type mice, the CRP^{-/-} mice of the instant invention demonstrated:
(1) 57% reduction in plasma TNF-α levels and 74% reduction in plasma IL-10 levels following LPS challenge,
(2) 35% reduction in INF-γ levels and 51% reduction in IL-2 levels following α-CD3 stimulation, and/or
(3) 50% increase in T-cell independent IgM antibody production after immunization with TNP-ficoll.

A skilled artisan will understand that owing to genetic and environmental factors, the aforementioned values are not absolute, but are indicative of the phenotypic differences. Thus the skilled worker may rely on one, two, three, or any combination of the aforementioned phenotypic differences to characterize the knockout animal of the present invention. Such phenotypic differences may be employed independently or together with the hereinbefore described genetic screening techniques (for example, mRNA or protein expression studies) for characterizing the knockout animal of the present invention.

The instant invention also relates to organs, tissues, cells, cell-lines, or sub-cellular fractions derived from CRP knockout animals of the present application. Preferably, such components are derived from animals which are homozygous for the CRP knockout genotype (CRP^{-/-}).

Examples of organs include, but are not limited to, spleen, thymus, liver, pancreas, heart, lung, kidney, bladder, brain, or blood.

Examples of tissues include, but are not limited to, muscle tissue, connective tissue, nerve tissue, or epithelial tissue.

Examples of cells include, but are not limited to, gamete cells (i.e., eggs, sperm), spleenocytes, thymus cells, blood cells, epithelial cells, hepatic cells, pancreatic cells, cardiomyocytes, or nerve cells. Also included are stem cells of embryonic or adult lineage.

Examples of cell-lines include, but are not limited to, primary cells, transformed cells, as well as immortalized cells.

The gene disruption, as used herein, may comprise one or more mutations in either the regulatory sequence CRP or in coding sequence thereof. Possible outcomes may include, for example, an untranslated gene product (no protein) or an incompletely translated gene product (mutant protein). “Mutation” as used herein may thus result in total or partial loss of CRP gene function.

The present invention also provides methods of producing a non-human animal that lacks a functional CRP gene, or a homolog thereof.

Preferably, the animal is a mammal.

In one embodiment there is provided a method for obtaining a CRP knockout mammal comprising crossing a transgenic mammal having a CRP gene or an exon thereof flanked with recognition sites for a site specific recombination enzyme with a transgenic animal expressing a constitutively active or inducible recombinase. Such methods are known in the art, and a representative example is provided below.

Briefly, the standard methodology for producing a knockout embryo requires introducing a targeting construct, which is designed to integrate by homologous recombination with the endogenous nucleic acid sequence of the targeted gene, into a suitable embryonic stem cell (ES). The ES cells are then cultured under conditions that allow for homologous recombination (i.e., of the recombinant nucleic acid sequence of the targeting construct and the genomic nucleic acid sequence of the host cell chromosome). Genetically engineered stem cells that are identified as comprising a knockout genotype that comprises the recombinant allele are introduced into an animal, or parent thereof, at an embryonic stage using standard techniques that are well known in the art (e.g., by microinjecting the genetically engineered embryonic stem (ES) cell into a blastocyst). The resulting chimeric blastocyst is then placed within the uterus of a pseudopregnant foster mother for the development into viable pups. The resulting viable pups include potentially chimeric founder animals whose somatic and germline tissue comprise a mixture of cells derived from the genetically-engineered ES cells and the recipient blastocyst. The contribution of the genetically altered stem cell to the germline of the resulting chimeric mice allows the altered ES cell genome, which comprises the disrupted target gene, to be transmitted to the progeny of these founder animals, thereby facilitating the production of “knockout animals” whose genomes comprise a gene that has been genetically engineered to comprise a particular defect in a target gene.

One of skill in the art will easily recognize that the CRP gene can be disrupted in a number of different ways, any one of which may be used to produce the CRP knockout animals of the present invention. For example, a knockout mouse according to the instant invention can be produced by the method of gene targeting. As used herein the term “gene targeting” refers to a type of homologous recombination that occurs as a consequence of the introduction of a targeting construct (e.g., vector) into a cell (e.g., an ES cell) that is designed to locate and recombine with a corresponding portion of the nucleic acid sequence of the genomic locus targeted for alteration (e.g., disruption) thereby introducing an exogenous recombinant nucleic acid sequence capable of conferring a planned alteration to the endogenous gene. Thus, homologous recombination is a process (e.g., method) by which a particular DNA sequence can by replaced by an exogenous genetically engineered sequence. More specifically, regions of the targeting vector that have been genetically engineered to be homologous or complementary to the endogenous nucleotide sequence of the gene that is targeted for transgenic disruption line up or recombine with each other such that the nucleotide sequence of the targeting vector is incorporated into (e.g., integrates with) the corresponding position of the endogenous gene.

The instant invention also relates to DNA sequences for creating the knockout animals of the instant invention and vectors derived therefrom. In one embodiment, there is provided a CRP DNA knockout construct comprising a selectable marker sequence flanked by DNA sequences homologous to the CRP gene of an animal, wherein when said construct is introduced into said animal at an embryonic stage, said selectable marker sequence disrupts the CRP gene in said mouse.

Additionally, the present invention provides a vector construct (e.g., a CRP targeting vector or CRP targeting construct) designed to disrupt the function of a wild-type (endogenous) CRP gene. In general terms, an effective CRP targeting vector comprises a recombinant sequence that is effective for homologous recombination with an endogenous CRP gene. For example, a replacement targeting vector comprising a genomic nucleotide sequence that is homologous to the target sequence operably linked to a second nucleotide sequence that encodes a selectable marker gene exemplifies an effective targeting vector. Integration of the targeting sequence into the chromosomal DNA of the host cell (e.g., embryonic stem cell) as a result of homologous recombination introduces an intentional disruption, defect or alteration (e.g., insertion, deletion or substitution) into the targeted sequence of the endogenous gene, e.g., the CRP gene. One aspect of the present invention is to replace all or part of the nucleotide sequence of a non-human gene that encodes the CRP polypeptide, thereby making a transgenic CRP knockout. A schematic example of such construct is shown in FIG. 1.

One of skill in the art will recognize that any CRP genomic nucleotide sequence of appropriate length and composition to facilitate homologous recombination at a specific site that has been preselected for disruption can be employed to construct a CRP targeting vector. Guidelines for the selection and use of sequences are described for example in Deng, C. and Capecchi, M., 1992, Mol. Cell. Biol., 12:3365-3371, and Bollag, R. et al., 1989, Annu. Rev. Genet., 23:199-225. For example, a wild-type CRP gene can be mutated and/or disrupted by inserting a recombinant nucleic acid sequence (e.g., a CRP targeting construct or vector) into all or a portion of the CRP gene locus. For example, a targeting construct can be designed to recombine with a particular portion within the enhancer, promoter, coding region, start codon, noncoding sequence, introns or exons of the CRP gene. Alternatively, a targeting construct can comprise a recombinant nucleic acid that is designed to introduce a stop codon after an exon of the CRP gene.

Suitable targeting constructs of the invention can be prepared using standard molecular biology techniques known to those of skill in the art. For example, techniques useful for the preparation of suitable vectors are described by Maniatis, et al., Molecular Cloning: A Laboratory Manual, Cold Spring Harbor Laboratory, Cold Spring Harbor, N.Y.; which disclosures are hereby incorporated by reference. Appropriate vectors include a replacement vector such as the insertion vector described by Capecchi, M., 1989, Science, 244:1288-92, which disclosure is hereby incorporated by reference; or a vector based on a promoter trap strategy or a polyadenylation trap, or “tag-and-exchange” strategy described by Bradley, et al., 1992, Biotechnology (NY), 10:534-539; and Askew, G. et al., 1993, Mol. Cell. Biol., 13:4115-4124, which disclosures are also incorporated herein by reference.

One of skill in the art will readily recognize that a large number of appropriate vectors known in the art can be used as the basis of a suitable targeting vector. In practice, any vector that is capable of accommodating the recombinant nucleic acid sequence required to direct homologous recombination and to disrupt the target gene can be used. For example, pBR322, pACY164, pKK223-3, pUC8, pKG, pUC19, pLG339, pR290, pKC101 or other plasmid vectors can be used. Alternatively, a viral vector such as the lambda gt11 vector system can provide the backbone (e.g. cassette) for the targeting construct.

The instant invention also relates to the use of the knockout animal of the instant invention, including components such as organs, tissues, cells, cell-lines, and/or sub-cellular fractions derived therefrom.

Preferably, in the instant invention, there is provided a method of using the knockout animal of the instant invention in screening for novel therapeutic and/or diagnostic agents.

In one embodiment, the instant invention relates to a method for screening for an immunomodulatory agent, comprising:
(a) administering said test compound to an experimental animal which is the CRP knockout animal of the instant invention;
(b) measuring the response of said experimental animal to said test compound;
(c) comparing the response of said experimental animal to a control animal; and
(d) selecting an agent based on the difference in response observed between said animal and said control animal.

In the instant invention, it was found that a CRP deficient (CRP^{-/-}) animal has differential cytokine production compared to an animal having a functional CRP gene. As shown in the figures, compared to wild-type mouse, plasma levels of certain cytokines (for example, IL-2, IL-10 and TNF-alpha) were attenuated while the levels of other cytokines (for example, IL-6) were elevated in the CRP deficient mouse.

Thus, in the instant invention, there is provided a method for screening for an immunomodulatory compound comprising measuring levels of one or more such cytokines. Preferably, the cytokine measured is a plasma cytokine.

Preferably, the immunomodulatory compound is an immunostimulant. However, the method could be adapted towards assaying for an immunosuppressant comprising measuring the levels of a different set of cytokines in the control and experimental animal.

As is known in the art, the control animal could be a CRP deficient (CRP^{-/-}) animal that has been administered a placebo compound, for example, buffer, salt, sugar, or a another non-toxic substance (i.e., negative control). Additionally, a positive control animal which is a CRP deficient (CRP^{-/-}) animal that has been administered a known immunomodulant (i.e., a known immunostimulant or immunosuppressant) could also be employed.

In a separate embodiment, wild type animals may also be employed as controls.

It was found that CRP-deficient animals stimulated with an inflammatory stimulus, for example, treatment with lipopolysaccharide (LPS), had attenuated levels of IL-2, IL-10 and TNF-α compared to wild-type animals. IL-6 production was elevated in CRP-deficient animals compared to wild-type animals.

In one embodiment, the hereinbefore-described assay relates to a method for screening for an immunostimulant comprising
(a) administering a test immunostimulant to an experimental animal which is the CRP knockout animal of the instant invention;
(b) measuring the level of at least one cytokine which is IL-2, IL-10, or TNF-α in said experimental animal;
(c) comparing the level of said IL-2, IL-10, or TNF-α in said experimental animal to a control animal; and
(d) selecting an agent based on the elevation of said IL-2, IL-10, or TNF-α in said experimental animal compared to said control animal.

Preferably, both the experimental as well as the control animals have been challenged with the inflammatory stimulus prior to administration of the test compound.

In another embodiment, the hereinbefore-described assay relates to a method for screening for an immunosuppressant comprising
(a) administering a test immunosuppressant to an experimental animal which is the CRP knockout animal of the instant invention;
(b) measuring the level of at least one cytokine which is IL-6 in said experimental animal;
(c) comparing the level of said IL-6 in said experimental animal to a control animal; and
(d) selecting an agent based on the attenuation of said IL-6 in said experimental animal compared to said control animal.

Particularly preferred experimental animals are mammals, wherein the plasma levels of one or more cytokines (for example, IL-2, IL-10, TNF-α and IL-6) are measured. Examples of such mammals include, but are not limited to, mouse, rat, cat, dog, cow, horses, etc.

Most preferably the hereinbefore described screening method is directed to methods (A) or (B):

(A) A method for screening for an immunostimulant comprising
(a) administering a test immunostimulant to an experimental animal which is an LPS-challenged CRP deficient (CRP^{-/-}) mouse;
(b) measuring the level of at least one cytokine which is IL-2, IL-10 or TNF-α in said experimental animal;
(c) comparing the level of at least one cytokine which is IL-2, IL-10 or TNF-α in said experimental animal to a control mouse; and
(d) selecting an agent based on the elevation of said IL-2, IL-10, or TNF-α in said experimental animal compared to said control mouse.

(B) A method for screening for an immunosuppressant comprising
(a) administering a test immunosuppressant to an experimental animal which is an LPS-challenged CRP deficient (CRP^{-/-}) mouse;
(b) measuring the level of IL-6 in said experimental animal;
(c) comparing the level of said IL-6 in said experimental animal to a control mouse; and
(d) selecting an agent based on the attenuation of said IL-6 in said experimental animal compared to said control mouse.

A skilled artisan will comprehend that organs, tissues, cells, cell-lines, and sub-cellular fractions derived from the animals of the instant invention may also be employed for desired in vitro assays.

The CRP knockout mouse of the instant invention is also useful for the in vivo study of the physiological outcome(s) of CRP deficiency and implications thereof, for example, in relation to the etiology of hereinbefore described diseases.

Transgenic Animals

With the knowledge of the cDNA encoding CRP and regulatory sequences regulating expression thereof, it is possible to generate transgenic animals, especially rodents, e.g., for testing the compounds which can alter CRP expression, translation or function in a desired manner. This procedure for transient over-expression in animals following infection with adenoviral vectors is described below in the examples.

A skilled worker understands that there are basically two types of animals which are useful in this regard: those not expressing functional CRP, and those which over-express CRP, either in those tissues which already express the protein or in those tissues where only low levels are naturally expressed.

The animals in the first group are preferably made using techniques that result in “knocking out” of the gene for CRP, although in the preferred case this will be incomplete, either only in certain tissues, or only to a reduced amount. These animals are preferably made using a construct that includes complementary nucleotide sequence to the CRP gene, but does not encode functional CRP, and is most preferably used with embryonic stem cells to create chimeras. Animals which are heterozygous for the defective gene can also be obtained by breeding a homozygote normal with an animal which is defective in production of CRP. These animals can then be crossed with other transgenic or knockout animals, as described in the following examples.

The animals in the second group are preferably made using a construct that includes a tissue specific promoter, of which many are available and described in the literature, or an unregulated promoter or one which is modified to increase expression as compared with the native promoter. The regulatory sequences for the CRP gene can be obtained using standard techniques based on screening of an appropriate library with the cDNA encoding CRP. These animals are most preferably made using standard microinjection techniques.

These manipulations are performed by insertion of cDNA or genomic DNA into the embryo using microinjection or other techniques known to those skilled in the art such as electroporation, as described in literature. The DNA is selected on the basis of the purpose for which it is intended: to inactivate the gene encoding a CRP or to overexpress or express in a different tissue the gene encoding CRP. The CRP encoding gene can be modified by homologous recombination with a DNA for a defective CRP, such as one containing within the coding sequence an antibiotic marker, which can then be used for selection purposes.

Animal Sources

Animals suitable for transgenic experiments can be obtained from standard commercial sources. These include animals such as mice and rats for testing of genetic manipulation procedures, as well as larger animals such as pigs, cows, sheep, goats, and other animals that have been genetically engineered using techniques known to those skilled in the art. These techniques are briefly summarized below based principally on manipulation of mice and rats.

Microinjection Procedures

The procedures for manipulation of the embryo and for microinjection of DNA are described in detail in Hogan et al. “Manipulating the mouse embryo,” Cold Spring Harbor Laboratory, Cold Spring Harbor, N.Y. (1986), the teachings of which are incorporated herein. These techniques are readily applicable to embryos of other animal species, and, although the success rate may differ, it is considered to be a routine practice to those skilled in this art.

Transgenic Animals

Female animals are induced to superovulate using methodology adapted from the standard techniques used with mice. Randomly cycling adult females are mated with vasectomized males to induce a false pregnancy, at the same time as donor females. At the time of embryo transfer, the recipient females are anesthetized and the oviducts are exposed by an incision through the body wall directly over the oviduct. The ovarian bursa is opened and the embryos to be transferred are inserted into the infundibulum. After the transfer, the incision is closed by suturing.

Embryonic Stem (ES) Cell Methods

Introduction of cDNA into ES cells:

Methods for the culturing of ES cells and the subsequent production of transgenic animals, the introduction of DNA into ES cells by a variety of methods such as electroporation, calcium phosphate/DNA precipitation, and direct injection are described in detail in Teratocarcinomas and embryonic stem cells, a practical approach, ed. E. J. Robertson, (IRL Press 1987), the teachings of which are incorporated herein. Selection of the desired clone of transgene-containing ES cells is accomplished through one of several means. In cases involving sequence specific gene integration, a nucleic acid sequence for recombination with the CRP gene or sequences for controlling expression thereof is co-precipitated with a gene encoding a marker such as neomycin resistance. Transfection is carried out by one of several methods described in detail in Potter et al Proc. Natl. Acad. Sci. USA 81, 7161 (1984). Calcium phosphate/DNA precipitation, direct injection, and electroporation are the preferred methods. In these procedures, a number of ES cells are plated into tissue culture dishes and transfected with a mixture of the linearized nucleic acid sequence and a transfection reagent. The cells are fed with selection medium supplemented with an antibiotic such as G418 (between 200 and 500 pg/ml). Colonies of cells resistant to the antibiotic are isolated using cloning rings and expanded. DNA is extracted from drug resistant clones and Southern blotting experiments using the nucleic acid sequence as a probe are used to identify those clones carrying the desired nucleic acid sequences. In some experiments, PCR methods are used to identify the clones of interest.

DNA molecules introduced into ES cells can also be integrated into the chromosome through the process of homologous recombination, described by Capecchi, (1989). Direct injection results in a high efficiency of integration. Desired clones are identified through PCR of DNA prepared from pools of injected ES cells. Positive cells within the pools are identified by PCR subsequent to cell cloning (Zimmer and Gruss, Nature 338, 150-153 (1989)). DNA introduction by electroporation is less efficient and requires a selection step. Methods for positive selection of the recombination event (i.e., neo resistance) and dual positive-negative selection (i.e., neo resistance and ganciclovir resistance) and the subsequent identification of the desired clones by PCR have been described by Joyner et al., Nature 338, 153-156 (1989) and Capecchi, (1989), the teachings of which are incorporated herein.

Embryo Recovery and ES Cell Injection

Naturally cycling or superovulated females mated with males are used to harvest embryos for the injection of ES cells. Embryos of the appropriate age are recovered after successful mating. Embryos are flushed from the uterine horns of mated females and placed in Dulbecco's modified essential medium plus 10% calf serum for injection with ES cells. Approximately 10-20 ES cells are injected into blastocysts using a glass microneedle.

Transfer of Embryos to Pseudopregnant Females

Randomly cycling adult females are paired with vasectomized males. Recipient females are mated such that they will be at 2.5 to 3.5 days post-mating (for mice, or later for larger animals) when required for implantation with blastocysts containing ES cells. At the time of embryo transfer, the recipient females are anesthetized. The ovaries are exposed by making an incision in the body wall directly over the oviduct and the ovary and uterus are externalized. A hole is made in the uterine horn with a needle through which the blastocysts are transferred. After the transfer, the ovary and uterus are pushed back into the body and the incision is closed by suturing. This procedure is repeated on the opposite side if additional transfers are to be made.

Identification of Transgenic Animals

Samples (for example, 1-2 cm of tails) are removed from young animals. For larger animals, blood or other tissue can be used. To test for chimeras in the homologous recombination experiments, i.e., to look for contribution of the targeted ES cells to the animals, coat color has been used in mice, although blood could be examined in larger animals. DNA is prepared and analyzed by both Southern blot and PCR to detect transgenic founder (F0) animals and their progeny (F1 and F2).

Once the transgenic animals are identified, lines are established by conventional breeding and used as the donors for tissue removal and implantation using standard techniques which are well known in the art. Currently, the most frequently used techniques for generating chimeric and transgenic animals are based on genetically altered embryonic stem cells or embryonic germ cells. Techniques suitable for obtaining transgenic animals have been amply described in the art. A suitable technique for obtaining completely ES cell derived transgenic non-human animals is described in WO 98/06834, the teachings of which are incorporated herein in its entirety.

Recombinases:

Preferably, the instant invention provides methods for obtaining a CRP knockout mouse of the instant invention using embryonic stem (ES) cell technology. The features of suitable preferred methods for obtaining the CRP knockout mice of the invention are, on the one hand, that the CRP gene is flanked with recognition sites for a site specific recombination enzyme (recombinase), and that, on the other hand, the recombinase can be provided by crossing the conditional knock-out mouse with a transgenic mouse expressing a constitutively active or inducible recombinase in the tissue of interest, i.e. the liver. Liver-specific expression can be achieved by using a promoter specific for liver cells, in particular hepatocytes. Examples for suitable promoters are known in the art.

Bacteriophage P1 Cre recombinase and flp recombinase from yeast plasmids are two non-limiting examples of site-specific DNA recombinase enzymes which cleave DNA at specific target sites (lox P sites for cre recombinase and frt sites for flp recombinase) and catalyze a ligation of this DNA to a second cleaved site. A large number of suitable alternative site-specific recombinases have been described, and their genes can be used in accordance with the method of the present disclosure. Such recombinases include the Int recombinase of bacteriophage λ (with or without Xis) (Weisberg, R. et. al., in Lambda II, (Hendrix, R., et al., Eds.), Cold Spring Harbor Press, Cold Spring Harbor, N.Y., pp. 211-50 (1983), herein incorporated by reference); TpnI and the β-lactamase transposons (Mercier, et al., J. Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J. Bacteriol., 172:610-18 (1990)); the B. subtilis SpolVC recombinase (Sato, et al., J. Bacteriol. 172:1092-98 (1990)); the Flp recombinase (Schwartz & Sadowski, J. Molec. Biol., 205:647-658 (1989); Parsons, et al., J. Biol. Chem., 265:4527-33 (1990); Golic & Lindquist, Cell, 59:499-509 (1989); Amin, et al., J. Molec. Biol., 214:55-72 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol., 205:493-500 (1989)), all herein incorporated by reference. Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al. (Mol Cell. Biochem., 92:107-16 (1990)); and Cregg & Madden (Mol. Gen. Genet., 219:320-23 (1989)), all herein incorporated by reference.

Cre has been purified to homogeneity, and its reaction with the loxP site has been extensively characterized (Abremski & Hess J. Mol. Biol. 259:1509-14 (1984), herein incorporated by reference). Cre protein has a molecular weight of 35,000 and can be obtained commercially from New England Nuclear/Du Pont. The cre gene (which encodes the Cre protein) has been cloned and expressed (Abremski, et al. Cell 32:1301-11 (1983), herein incorporated by reference). The Cre protein mediates recombination between two loxP sequences (Sternberg, et al. Cold Spring Harbor Symp. Quant. Biol. 45:297-309 (1981)), which may be present on the same or different DNA molecule. Because the internal spacer sequence of the loxP site is asymmetrical, two loxP sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al. Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

Recombinases have important application for characterizing gene function in knockout models. When the constructs described herein are used to disrupt target genes, a fusion transcript can be produced when insertion of the positive selection marker occurs downstream (3′) of the translation initiation site of the target gene. The fusion transcript could result in some level of protein expression with unknown consequence. It has been suggested that insertion of a positive selection marker gene can affect the expression of nearby genes. These effects may make it difficult to determine gene function after a knockout event since one could not discern whether a given phenotype is associated with the inactivation of a gene, or the transcription of nearby genes. Both potential problems are solved by exploiting recombinase activity. When the positive selection marker is flanked by recombinase sites in the same orientation, the addition of the corresponding recombinase will result in the removal of the positive selection marker. In this way, effects caused by the positive selection marker or expression of fusion transcripts are avoided.

In a preferred embodiment, the knockout construct of the instant invention comprises a recognition site which is LoxP and utilizes a Cre recombinase. The recombinase may be placed under the transcriptional control of a constitutively active promoter or a tissue-specific promoter.

Deletion of the CRP gene in a tissue-specific or time-specific manner may be achieved using art known techniques. An inducible gene deletion system enabling to delete both genes in adult mice, as described by Vasioukhin et al. (1999) may also be used.

For obtaining the tissue-specific knock-out mice of the invention, according to a preferred embodiment, the hereinbefore described inducible loxP/Cre system is used. To date, this system is considered to be the most reliable experimental setup for spatio-temporally controlled site-specific somatic gene deletion in vivo. The deletion of the gene(s) of interest (in the case of the present invention CRP) can be induced either by systemic injection or local application of an inducing agent. Such techniques are known in the art (Vasioukhin et al., 1999).

Alternatively to the loxP/Cre-system, other spatio-temporally controlled site-specific somatic gene deletion systems can be used to generate tissue-specific knock-out mice of the instant invention. Examples for such alternative methods for engineering the conditional knock-out mice of the invention are the Flp-FRT and the phiC31-att site-specific recombinase systems. As the loxP/Cre-system, these systems fulfill the requirements of having the gene(s) of interest flanked with recognition sites for the site specific recombination enzyme and of providing the recombination enzyme by crossing the conditional knock-out mouse with a transgenic mouse expressing a constitutively active or inducible recombinase in the tissue of interest (Branda and Dymecki, 2004).

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus.

The term “transgenic animal” is not meant to encompass classical cross-breeding or in vitro fertilization, but rather is meant to encompass animals in which one or more cells are altered by or receive a recombinant DNA molecule. This molecule may be specifically targeted to defined genetic locus, be randomly integrated within a chromosome, or it may be extrachromosomally replicating DNA.

The term “germ cell line transgenic animal” refers to a transgenic animal in which the genetic alteration or genetic information was introduced into a germ line cell, thereby conferring the ability to transfer the genetic information to offspring. If such offspring in fact possess some or all of that alteration or genetic information, they are transgenic animals as well. Methods for generating transgenic animals via embryo manipulation and microinjection, particularly animals such as mice, have become conventional in the art and are described, for example, in U.S. Pat. Nos. 4,736,866; 4,870,009; 4,873,191; and in Hogan, B., Manipulating the Mouse Embryo, (Cold Spring Harbor Laboratory Press, Cold Spring Harbor, N.Y., 1986), herein incorporated by reference in their entirety. Similar methods are used for production of other transgenic animals. A transgenic animal can be produced by introducing nucleic acid into the male pronuclei of a fertilized oocyte, e.g., by microinjection, retroviral infection, and allowing the oocyte to develop in a pseudopregnant female foster animal. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes.

As used herein, the term “gene” refers to DNA sequences that encode the genetic information (e.g., nucleic acid sequence) required for the synthesis of a single protein (e.g., polypeptide chain). In addition to the “coding sequence,” the sequence that directly codes the amino acid sequence, a gene also includes essential non-coding elements, e.g., promoters, enhancers, silencers, and non-essential flanking and intron sequences. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

The term “CRP gene” refers to a particular gene that comprises a DNA sequence that encodes the CRP protein.

As is understood by one of skill in the art, a gene sequence can contain “sites” (sequence positions) that are different among individuals in a population. Thus, a gene allows for variation of the sequence. Each variant sequence is referred to as an “allele” of the gene. Therefore, as used herein, the term “allele” refers to any of several alternative forms of a gene.

Typically, a particular sequence, usually one that encodes a functional protein, is taken to be a reference or “wild-type” sequence; the term “wild-type” is a descriptive term meant to connote a reference allele, typically an allele that encodes a functional protein or an allele present in a healthy individual. Alleles that differ from the wild-type sequence are referred to as “allelic variants”. Homologous chromosomes are chromosomes that pair during meiosis and contain substantially identical loci. The term “locus” connotes the site (e.g., location) of a gene on a chromosome.

The term “homolog” refers to a gene similar in structure and evolutionary origin to a given gene.

The term “germ-line” refers to a condition wherein genetic alteration or genetic variation was introduced into a germ line cell, thereby conferring the ability to transfer the genetic information to offspring. If such offspring in fact, possess some or all of that alteration or genetic variation, then they, too, are transgenic animals.

As readily understood by those of skill in the art, the term “global” or “total” in reference to a transgenic animal means that the genetic modification is present in all cells. Similarly, “tissue specific” refers to the substantially exclusive initiation of transcription in the tissue from which a particular promoter drives expression of a given gene.

The alteration or genetic information may be foreign to the species of animal to which the recipient belongs, or foreign only to the particular individual recipient, or may be genetic information already possessed by the recipient. In the last case, the altered or introduced gene may be expressed differently than the native gene.

“Gene targeting” is a type of homologous recombination that occurs when a fragment of genomic DNA is introduced into a cell and that fragment locates and recombines with endogenous homologous sequences.

A “knockout mouse” is a mouse that contains within its genome a specific gene that has been inactivated by the method of gene targeting. A knockout mouse includes both the heterozygote mouse (i.e., one defective allele and one wild-type allele) and the homozygous mutant (i.e., two defective alleles).

A “mutation” is a detectable change in the genetic material in the animal, which is transmitted to the animal's progeny. A mutation is usually a change in one or more deoxyribonucleotides, the modification being obtained by, for example, adding, deleting, inverting, or substituting for nucleotides.

A “cell line” is a permanently established specific cell culture that will proliferate indefinitely given appropriate medium and conditions. The cell-line can also be fractionated into “sub-cellular” fractions where, for example, the receptor can be found. For example, cells expressing the receptor can be fractionated into the nuclei, the endoplasmic reticulum, vesicles, or the membrane surfaces of the cell.

As used herein, the term “vector” refers to nucleic acid sequences, arranged in such an order and containing appropriate components such that they are taken up into cells or can be inserted into cells through microinjection or other techniques. Such sequences may or may not naturally be present in the cell, either in whole or in part. Typically, the vector contains a promoter or promoters, a structural gene of interest that is to be transferred and expressed in the cell or organism (host) transfected with the vector, and other elements necessary for gene transfer and/or expression in the host such as sequences enabling the processing and translation of the transcription sequences, including translation initiation and polyadenylation sequences. In the present invention, the vector used may be circular or linear, and is preferably linear for insertion into embryos to generate a transgenic mammal.

A “marker gene” is a selection marker that facilitates the isolation of rare transfected cells from the majority of treated cells in the population. A non-comprehensive list of such markers includes neomycin phophotransferase, hygromycin B phophotransferase, Xanthiline/guanine phosphoribosyl transferase, herpes simplex thymidine kinase, and diphtheria toxin.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1. Schematic representation of targeted deletion of the CRP gene used to generate the CRP^{-/-} mice, wherein exon 2 is deleted from the endogenous CRP gene through the action of Cre-recombinase of the floxed allele.

FIG. 2. Expression of CRP mRNA was absent in liver lysates from three CRP^{-/-} mice compared to wild type controls. qRT-PCR was used to determine mRNA expression based on primers designed for mouse CRP.

FIG. 3. Western blot analysis using an anti-mouse CRP antibody on liver lysates. The lysates from three CRP^{-/-} mice showed no expression of CRP protein compared to lysates from three wild type controls.

FIG. 4. Panels (A) and (B). LPS-induced plasma TNF-alpha cytokine production in CRP deficient (CRP^{-/-}) mice.

FIG. 5. LPS-induced plasma IL-6 cytokine production in wild-type and CRP deficient (CRP^{-/-}) mice.

FIG. 6. Panels (A) and (B). LPS-induced plasma IL-10 cytokine production in wild-type and CRP deficient (CRP^{-/-}) mice.

FIG. 7 Shows TNP-Ficoll induced anti-TNP IgM production in CRP deficient (CRP^{-/-}) mice.

FIG. 8: Panels (A) and (B). anti-CD3 antibody-induced plasma interferon-gamma (IFNγ) cytokine production in CRP deficient (CRP^{-/-}) mice.

FIG. 9: anti-CD3 antibody-induced plasma interferon-gamma (IFNγ) cytokine production in splenocytes obtained from wild-type and CRP-deficient (CRP^{-/-}) mice. SEB and ConA were used as controls.

FIG. 10: Panel (A). anti-CD3 antibody-induced plasma interleukin-2 (IL-2) cytokine levels in wild-type and CRP deficient (CRP^{-/-}) mice.

FIG. 11: anti-CD3 antibody-induced plasma interleukin-2 (IL-2) cytokine levels in splenocytes obtained from wild-type and CRP-deficient (CRP^{-/-}) mice. SEB and ConA were used as controls.

Without further elaboration, it is believed that one skilled in the art, using the preceding description, can utilize the present invention to its fullest extent. The following preferred specific embodiments are, therefore, to be construed as merely illustrative, and not limitative of the remainder of the disclosure in any way whatsoever. The entire disclosure of all applications, patents and publications, cited above and in the figures are hereby incorporated by reference in their entirety.

In the forgoing and in the following examples, all temperatures are set forth uncorrected in degrees Celsius and, all parts and percentages are by weight, unless otherwise indicated.

### EXAMPLES

The invention will be explained below with reference to the following non-limiting examples.

### Example 1

Animals. All animal experiments were performed in accordance with internal protocols established by Institutional Animal Care and Use Committee and under NIH guidelines.

Generation of conditionally mutant CRP mice. CRP mutant mice were generated in collaboration with Lexicon Genetics, Inc. The conditional targeting vector was derived using the Lambda KOS system. Mice heterozygous for loxP flanked exon 2 were bred with a protamine-Cre recombinase transgenic line. PCR primers were used for genotyping. Primers BI.25-3 (5′-GAA GTA TCT GAC TCC TTG GG-3′) and BI.25-33 (5′-ATG TAA CCT GGG AGA GGA C-3′) will yield a 159-base pair fragment for the wild-type allele and a 243-base pair fragment for the floxed allele, whereas primers BI.25-33 and BI.25-27 (5′-AAA GGG AGA GTA TCA GAA CC-3′) will detect a 281-base pair fragment for the cre-excised allele. Mice heterozygous for the deleted exon2 were breed to generate homozygous knockout mice. Mice were maintained on sterile normal rodent diet and bottled water ad libitum. Mice at 8-20 weeks were used for analysis. Livers from three wild type (B6.129) and CRP^{-/-} mice were harvested, snap frozen in liquid nitrogen, homogenized, and lysed for qRT-PCR based on primers designed for CRP mRNA. The same lysates were used for gel electrophoresis and Western blot analysis using an anti-mouse CRP antibody to detect mouse CRP protein.

Immunoblotting. Livers from three wild type (B6.129) and CRP^{-/-} mice were harvested, snap frozen in liquid nitrogen, homogenized, and lysed for qRT-PCR based on primers designed for CRP mRNA. The same lysates were used for gel electrophoresis and Western blot analysis using an anti-mouse CRP antibody to detect mouse CRP protein.

LPS induced TNF-α and IL-10 production: Animals were administered 200 ng LPS L-2280) plus 1 mg d-galactosamine intravenously in 0.2 ml of pyrogen-free saline. One hour after LPS/D-galactosamine, each mouse was anesthetized via inhalation of isoflurane and bled by retro-orbital puncture. Blood was spun at 14000 rpm for ˜5 minutes and the plasma was collected and assayed for TNF-alpha, and IL-10 using commercial murine ELISA kits.

α-CD3 induced cytokine production: 1 μg hamster α-mouse CD3 was administered by intraperitoneal injection in 0.2 ml DPBS to stimulate the production of interleukin 2 (IL-2) and other cytokines. Three hours after the administration of α-CD3, mice were anesthetized with isoflurane inhalation and bled via retro-orbital puncture. Blood was centrifuged at 14000 rpm for ˜5 min, plasma collected and assayed for IL-2, IL-4 and interferon gamma (IFN-γ) using commercially purchased murine ELISA kit.

Cytokine production of splenocytes to various mitogenic stimuli in vitro: Splenocytes from unmanipulated wild type and knock out mice were centrifuged and re-suspended to 5×10⁶ cells/ml complete media. 200 μl/well (5×10⁶ cells) of this cell preparation was added to 96 well flat bottomed culture plates with one of the following stimuli: 1.25 μg/ml plate bound α-mouse CD3 antibody, 1.25 μg/ml soluble α-mouse CD3 antibody, 1.25 μg/ml Concanavalin A, 6.25 μg/ml SEB, 250 μg/ml LPS or 6.25 ng/ml phorbol 12-myristate 13-acetate (PMA) 625 ng/ml ionomycin. Plates were set up for 24, 48 or 72 hour cytokines and the cytokines were assayed using mouse IL-2, IL-4, and IFN-gamma ELISA kits.

T cell Independent antibody production using TNP-ficoll: Mice were pre-bled via retro-orbital puncture (background), and then injected intraperitoneally with 10 μg TNP-Ficoll. Seven days after challenge, mice were anaesthetized under inhaled isoflurane. Whole blood was collected via retro-orbital puncture, and the plasma analyzed for antibodies to TNP via an ELISA.

Results

Generation of Transgenic Animals.

CRP mutant mice were generated in collaboration with Lexicon Genetics, Inc (The Woodlands, Tex.). The conditional targeting vector was derived using the Lambda KOS system. The conditional targeting vector was derived using the Lambda KOS system (Wattler et. al. BioTechniques 26:1150-1160, 1999). The Lambda KOS phage library, arrayed into 96 superpools, was screened by PCR using exon 1 and 2-specific primers Crp-1 [5′-GCAGCATCCATAGCCATGG-3′] and Crp-3 [5′-GAAGTATCTGACTCCTTGGG-3′]. The PCR-positive phage superpools were plated and screened by filter hybridization using the 338 by amplicon derived from primers Crp-1 and Crp-3 as a probe. Two pKOS genomic clones, pKOS-68 and pKOS-83, were isolated from the library screen and confirmed by sequence and restriction analysis. Gene-specific arms having the following sequence were used:

(5′-AGGACCAGATGACCCTTGATCCCAAACTCTAC-3′)
and
(5′-GCAGGAGGTAGTATGGCTTGGATATGATTCTG-3′).

The gene-specific arms were appended by PCR to a yeast selection cassette containing the URA3 marker. The yeast selection cassette and pKOS-68 were co-transformed into yeast, and clones that had undergone homologous recombination to replace a 1688 by region containing exon2 with the yeast selection cassette were isolated. This 1688 by fragment was independently amplified by PCR and cloned into the intermediate vector pLF-Neo introducing flanking LoxP sites and a Neo selection cassette (Crp-pLFNeo). The yeast cassette was subsequently replaced with the Crp-pLFNeo selection cassette to complete the conditional Crp targeting vector that has exon2 flanked by LoxP sites. The Not I linearized targeting vector was electroporated into 129/SvEvBrd (Lex-1) ES cells. G418/FIAU resistant ES cell clones were isolated, and correctly targeted clones were identified and confirmed by Southern analysis using a 278 by 5′ external probe (30/29), generated by PCR using primers Crp-30 [5′-CTTCAAAGCCTCTCAATTGCT-3′] and Crp-29 [5′-TTGTATTGCTCTGCCAGTCAA-3′], and a 284 by 3′ external probe (31/32), amplified by PCR using primers Crp-31 [5′-GGAGGTAGTTCCAATTTTGG-3′] and Crp-32 [5′-AAAGGATGTGACTAGCTTGG-3′]. Southern analysis using probe 30/29 detected a 15.7 Kb wild type band and 17.7 Kb mutant band in Nhe I digested genomic DNA while probe 31/32 also detected a 15.7 Kb wild type band and 17.7 Kb mutant band in Nhe I digested genomic DNA. Two targeted ES cell clones were microinjected into C57BU6 (albino) blastocysts. The resulting chimeras were mated to C57BU6 (albino) females to generate mice that were heterozygous for the Crp conditional mutation. Exon 2 was deleted by crossing these mice with a protamine-Cre recombinase transgenic line (O′Gorman et. al. PNAS 94: 14602-14607, 1997). 3 PCR primers were used for genotyping. Primers B1.25-3 (5′-GAA GTA TCT GAC TCC TTG GG-3′) and B1.25-33 (5′-ATG TAA CCT GGG AGA GGA C-3′) will yield a 159-base pair fragment for the wild-type allele and a 243-base pair fragment for the floxed allele, whereas primers B1.25-33 and B1.25-27 (5′-AAA GGG AGA GTA TCA GAA CC-3′) will detect a 281-base pair fragment for the cre-excised allele. Mice heterozygous for the deleted exon2 were breed to generate homozygous knockout mice. Mice were maintained on sterile normal rodent diet (PicoLab rodent 20 from LabDiet, Richmond, Ind.) and bottled water ad libitum. Mice at 8-20 weeks were used for analysis.

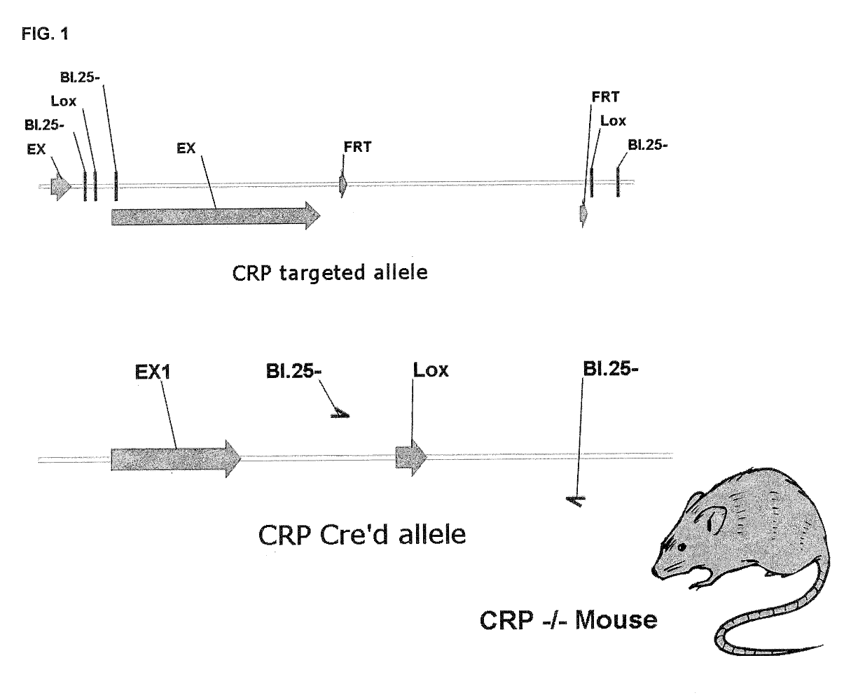

A schematic diagram is shown in FIG. 1.

Analysis of CRP expression in tissue samples:

Expression studies were conducted using TAQMAN probes as per the manufacturer's instructions. The TAQMAN assay-on-demand mouse CRP probes were ordered from ABI (Applied Biosystem, Inc.) comprising the following probes:

Mm02601590_g1 (CRP1)

Mm00432680_g1 (CRP2)

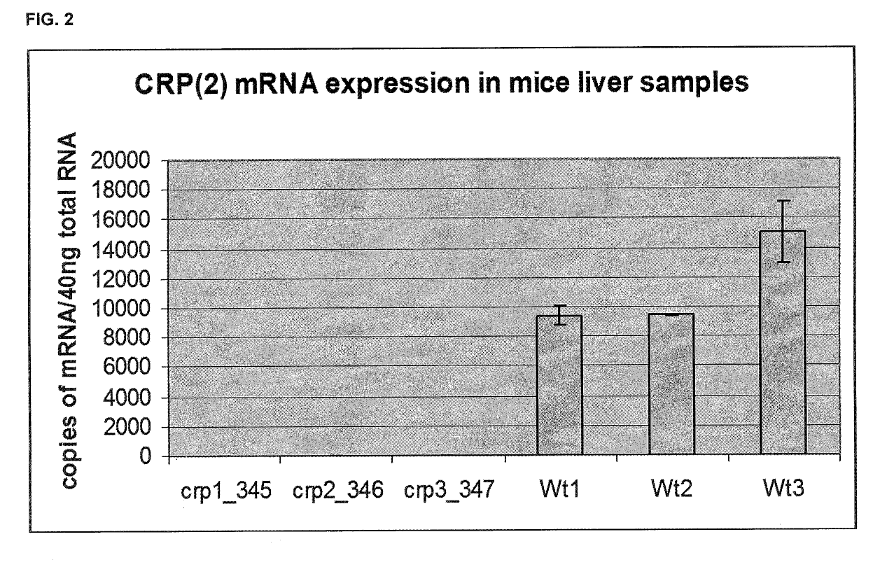

Both the probe sequences are designed in exon-intron boundary. Following probing for gene expression, values were normalized to mouse GAPDH levels (using the probe Mm99999915_g1). The results are shown in FIG. 2. Tissue samples obtained from homozygous knockout animals had almost undetectable CRP expression when compared to identical tissue samples obtained from wild-type animals. These studies indicate that transgenic CRP knockout animals were deficient in CRP gene at the genetic level.

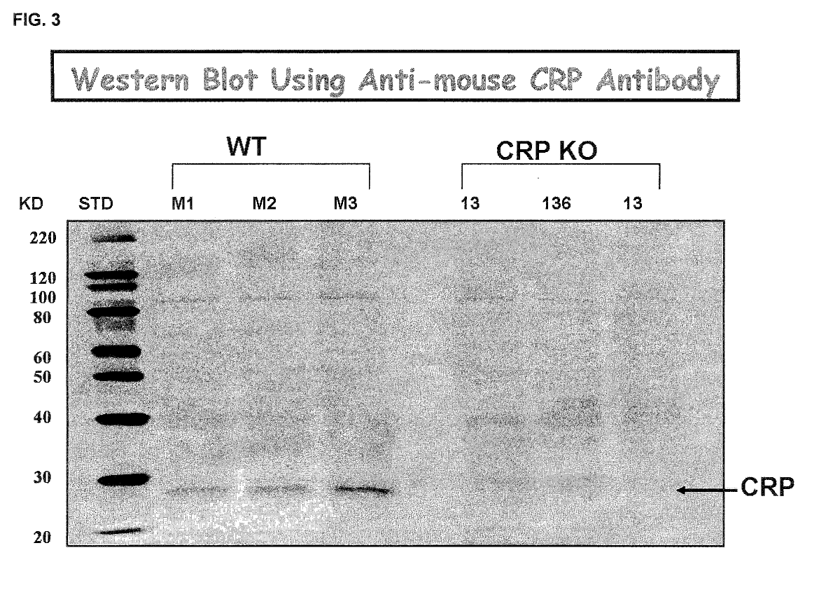

These studies were confirmed at the protein level using immunoblotting analysis. Livers from three wild type (B6.129) and CRP^{-/-} mice were harvested, snap frozen in liquid nitrogen, homogenized, and lysed for qRT-PCR based on primers designed for CRP mRNA. The same lysates were used for gel electrophoresis and Western blot analysis using an anti-mouse CRP antibody to detect mouse CRP protein. The results are shown in FIG. 3. CRP protein was consistently expressed in the livers of wild-type mice while liver cells obtained from homozygous knockout animals did not have measurable expression of the protein. These studies indicate that the transgenic CRP knockout animals were deficient in expression of CRP protein.

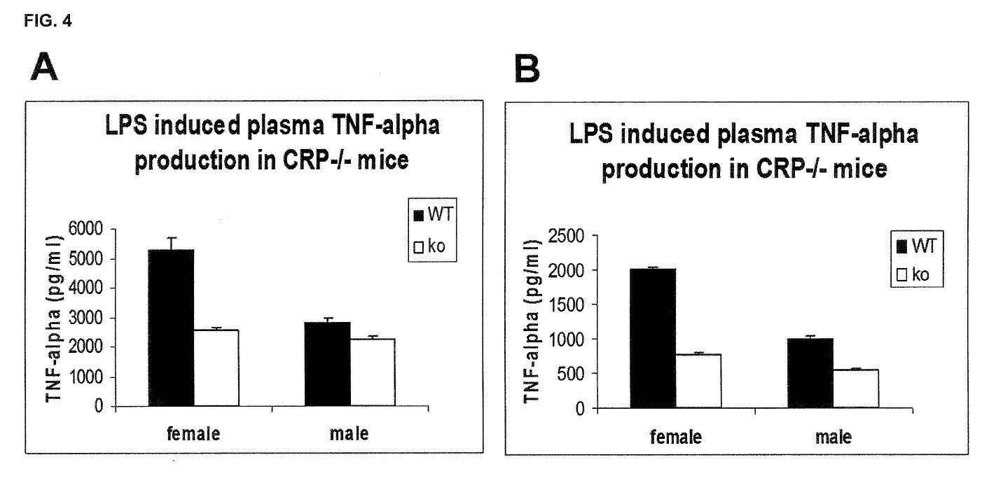

To study the phenotype of the CRP knockout animals, a host range of characterizations were performed. In one study, CRP deficient (CRP^{-/-}) mice were challenged with LPS and plasma TNF-alpha cytokine levels were analyzed. It was found that compared to wild type animals, CRP^{-/-} mice showed significantly reduced TNFα production following LPS stimulation in vivo. Both female (n=5) and male (n=8) mice showed reduced levels. Data represented as Mean+/−SEM (p<0.05# versus wild type). Results are presented in FIGS. 4 (A) and (B).

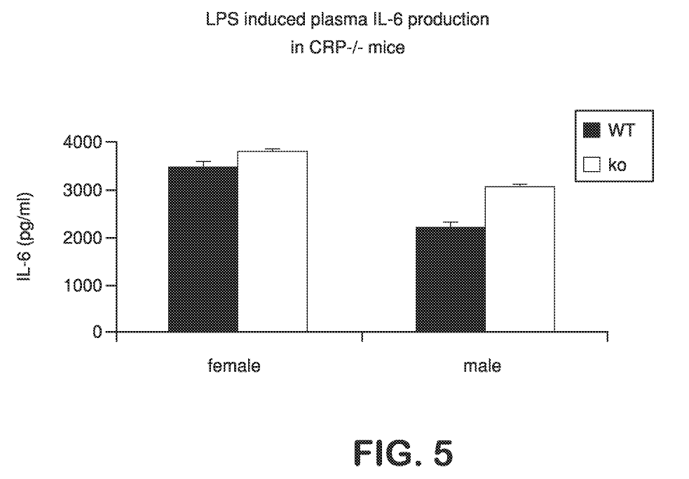

Additional differences between the immunological phenotype of wild type animals and CRP-deficient (CRP^{-/-}) animals were observed with respect to plasma IL-6 levels in LPS challenged animals. LPS induced plasma IL-6 cytokine production in CRP deficient (CRP^{-/-}) mice. CRP^{-/-} mice showed significantly increased IL-6 production following LPS stimulation in vivo. Both female and male mice showed elevated levels. Results are shown in FIG. 5.

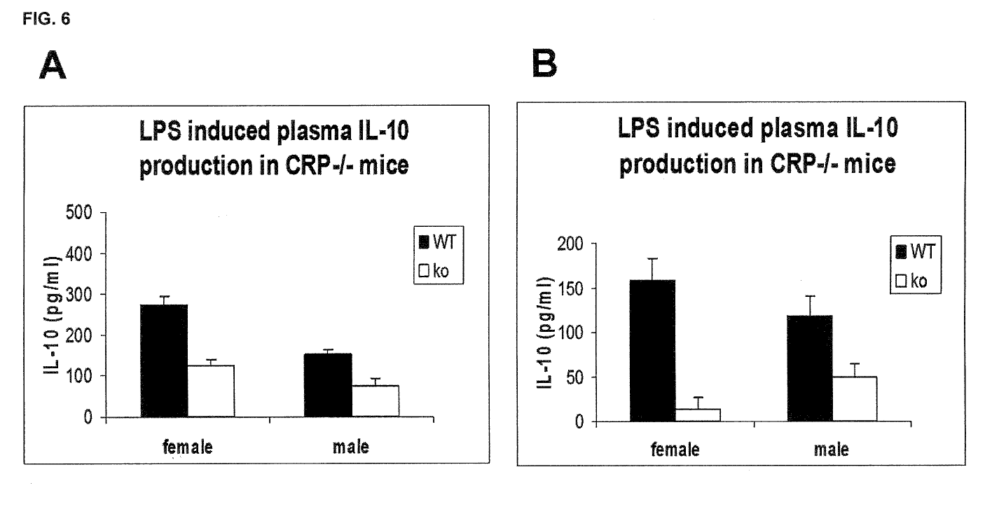

It was additionally found that CRP deficient (CRP^{-/-}) mice showed significantly reduced IL-10 production following LPS stimulation in vivo. As shown in FIGS. 6 (A) and (B), both female (n=4) and male (n=8) mice showed reduced IL-10 levels compared to wild-type animals. The differences were significant. Data represented as Mean+/−SEM (p<0.05# versus wild type).

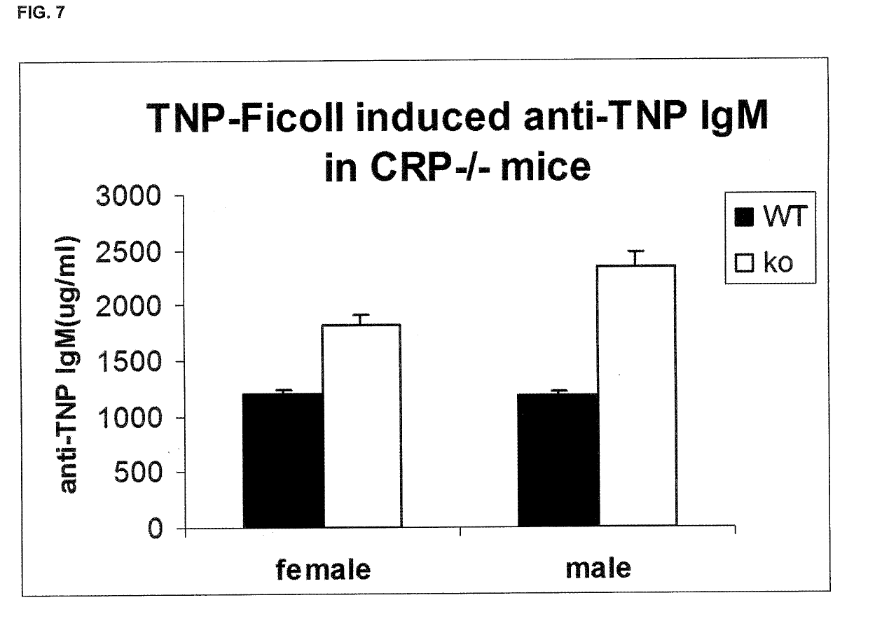

As can be seen from the results shown in FIG. 7, T-cell independent antibody production (IgM) was significantly increased following immunization with TNP ficoll in CRP^{-/-} mice compared to wild type.

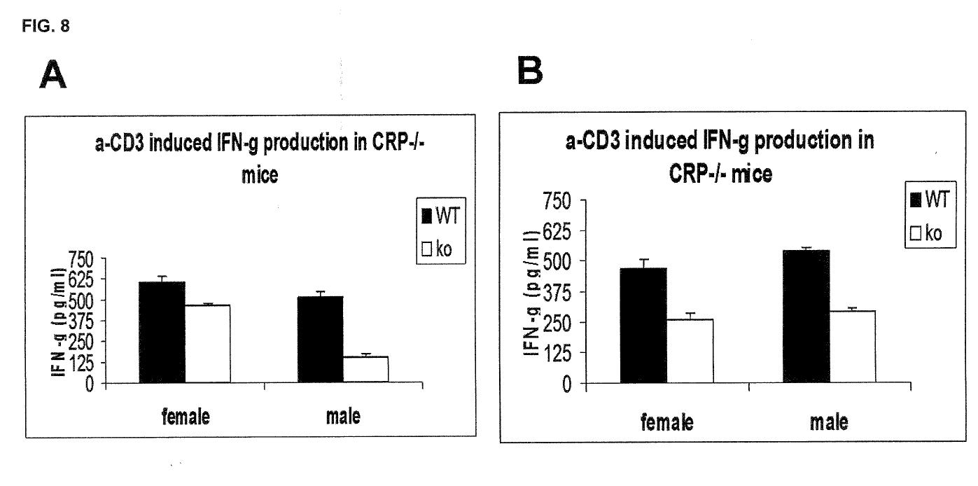

The next step was to analyze the effect of anti-CD3 antibody in wild-type and CRP-deficient animals. As shown in FIG. 8, panels (A) and (B), exposure to anti-CD3 antibody reduced plasma interferon-gamma (IFNγ) cytokine production in CRP deficient (CRP^{-/-}) mice. CRP^{-/-} mice showed significantly reduced INFγ production following anti-CD3 stimulation in vivo. Both female (n=4) and male (n=4) mice showed reduced levels.

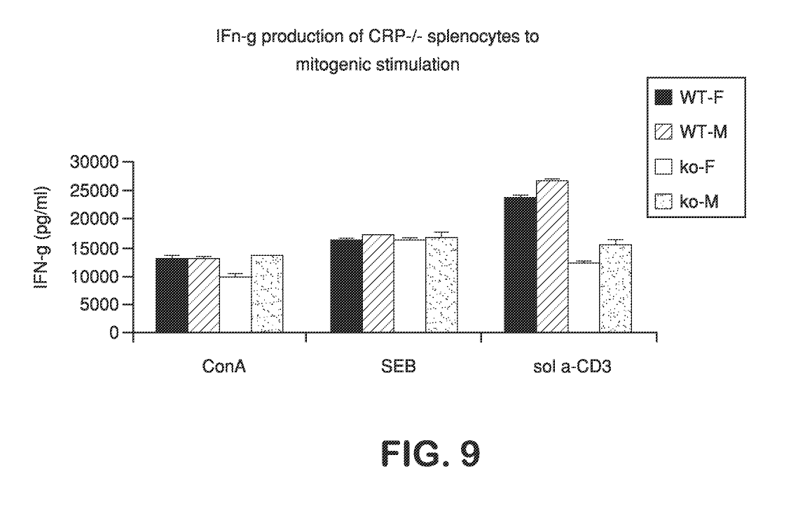

A similar study was conducted in vitro using splenocytes, the results of which are presented in FIG. 9. As shown in the figure, splenocytes treated with anti-CD3 antibody had greater interferon-gamma (IFNγ) cytokine production in splenocytes obtained from CRP-deficient (CRP^{-/-}) mice. Splenocytes from CRP^{-/-} mice showed significantly reduced INFγ production to anti-CD3 stimulation in vitro compared to wild type mice. Splenocytes from both female (n=4) and male (n=4) mice showed reduced levels. The decrease was specific for anti-CD3 stimulation as both SEB and ConA induced activation showed no difference in INFγ production. Data represented as Mean+/−SEM (p<0.05# versus wild type).

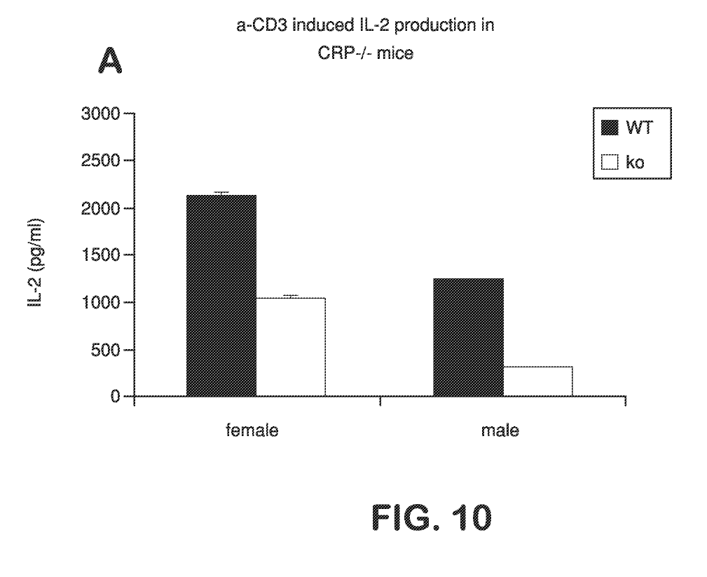

Next, the effect of anti-CD3 antibody on plasma interleukin-2 (IL-2) cytokine levels was examined in wild-type and CRP deficient (CRP^{-/-}) mice. CRP^{-/-} mice showed significantly reduced IL-2 production following anti-CD3 stimulation in vivo. Both female (n=6) and male (n=6) mice showed reduced levels. The results are shown in FIG. 10.

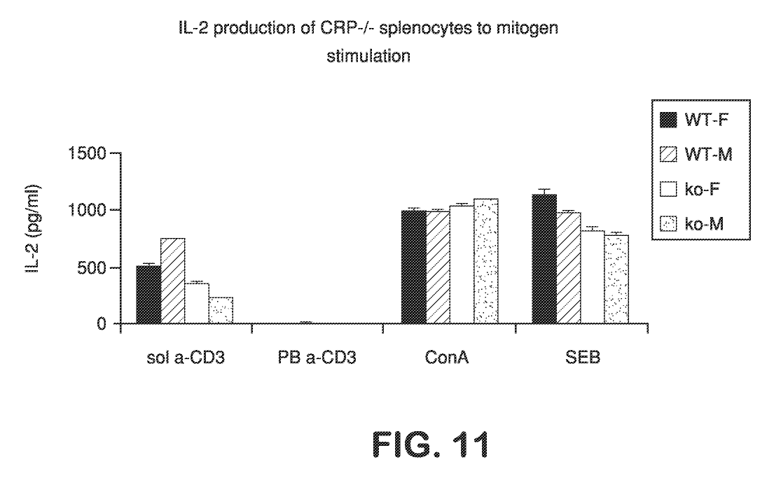

The effect of anti-CD3 antibody on plasma interleukin-2 (IL-2) cytokine production was analyzed in vitro using splenocytes derived from wild-type and CRP deficient (CRP^{-/-}) mice was examined. It was found that anti-CD3 antibody-induced plasma interleukin-2 (IL-2) cytokine levels in splenocytes obtained from CRP-deficient (CRP^{-/-}) mice. Splenocytes from CRP^{-/-} mice showed significantly reduced IL-2 production to anti-CD3 stimulation in vitro compared to wild type mice (FIG. 11). Splenocytes from both female (n=4) and male (n=4) mice showed reduced levels. The decrease was specific for anti-CD3 stimulation as both SEB and ConA induced activation showed no difference in INFγ production. Data represented as Mean+/−SEM (p<0.05# versus wild type).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

It is believed that one skilled in the art, using the preceding information and information available in the art, can utilize the present invention to its fullest extent. It should be apparent to one of ordinary skill in the art that changes and modifications can be made to this invention without departing from the spirit or scope of the invention as it is set forth herein. The topic headings set forth above and below are meant as guidance where certain information can be found in the application, but are not intended to be the only source in the application where information on such topic can be found. All publications and patents cited above are incorporated herein by reference.

We claim:

1. A transgenic animal which comprises a disrupted gene encoding a C-reactive protein (CRP) or a homolog thereof.

2. The animal of claim 1 which comprises a heterozygous or homozygous disruption of said CRP gene or said homolog thereof.

3. The animal of claim 1 which comprises a homozygous disruption for said CRP gene or said homolog thereof, wherein said homozygous disruption prevents the expression of a functional CRP protein or said homolog thereof in said animal.

4. The animal of claim 3, wherein said CRP gene or said homolog thereof is disrupted globally or in a tissue-specific manner.

5. The animal of claim 3, wherein said CRP gene or said homolog thereof is disrupted globally.

6. The animal of claim 3 which comprises a germ-line mutation or deletion of said CRP gene or said homolog thereof.

7. The animal of claim 3 which comprises a tissue-specific mutation or deletion of said CRP gene or said homolog thereof.

8. The animal of claim 3 which comprises homozygous disruption of one or more exons of said CRP gene or said homolog thereof.

9. The animal of claim 3 which comprises an altered phenotype compared to an animal having a wild type CRP gene, wherein said altered phenotype is:
(1) reduced cytokine production after LPS challenge;
(2) reduced T-cell cytokine production after α-CD3 stimulation;
(3) elevated T-cell independent antibody production after immunization with TNP-ficoll; or
(4) any combination of (1), (2) and (3).

10. The animal of claim 9, wherein said altered phenotype comprises
(1) reduced TNF-α and IL-10 cytokine production after LPS challenge;
(2) reduced INF-γ and IL-2 production after α-CD3 stimulation;
(3) elevated IgM antibody production; or
(4) any combination of (1), (2) and (3).

11. The animal of claim 3 which is a nematode, zebrafish, mouse, rat, guinea pig, rabbit, goat, sheep, cat, dog, or cow.

12. The animal of claim 3 which is a mouse.

13. An organ, a tissue, a cell, a cell-line, or a sub-cellular fraction derived from the animal of claim 1.

14. The cell or cell-line of claim 13 which is isolated from an embryo of said animal.

15. An organ, a tissue, a cell, a cell-line, or a sub-cellular fraction which is devoid of a functional expression of said CRP gene and which is derived from the animal of claim 3.

16. The organ, tissue, cell, cell-line or sub-cellular fraction of claim 15 which is derived from a liver of said animal.

17. A method for obtaining the animal of claim 3, wherein a transgenic animal having a CRP gene or an exon thereof flanked with recognition sites for a site specific recombination enzyme is crossed with a transgenic animal expressing a constitutively active or inducible recombinase.

18. The method of claim 16 wherein the recognition site is LoxP and the recombinase is Cre.

19. The method of claim 16 wherein the recombinase is under the transcriptional control of a tissue-specific promoter.

20. The method of claim 16 wherein the exon comprises the entire exon 2 of said CRP gene.

21. A CRP DNA knockout construct comprising a selectable marker sequence flanked by DNA sequences homologous to the CRP gene of an animal, wherein when said construct is introduced into said animal at an embryonic stage, said selectable marker sequence disrupts the CRP gene in said mouse.

22. A vector comprising the CRP DNA knockout construct of claim 21.

23. A CRP DNA knockout construct according to claim 21, which comprises a CRP targeted allele as depicted in FIG. 1.

24. A method for screening for an agent for the treatment of an inflammatory disease, comprising:
(a) administering said test compound to an experimental animal which is the animal of claim 3;
(b) measuring the response of said experimental animal to said test compound;
(c) comparing the response of said experimental animal to a control animal having a functional CRP gene; and
(d) selecting an agent based on the difference in response observed between said animal and said control animal.

25. The method of claim 24 wherein said experimental animal is a CRP knockout animal and said control animal comprises a wild-type or heterozygous deletion of said CRP gene.

26. A method for screening for an immunostimulant comprising
(a) administering a test immunostimulant to an experimental animal which is a CRP knockout animal;
(b) measuring the level of at least one cytokine which is IL-2, IL-10, or TNF-α in said experimental animal;
(c) comparing the level of said IL-2, IL-10, or TNF-α in said experimental animal to a control animal; and
(d) selecting an agent based on the elevation of said IL-2, IL-10, or TNF-α in said experimental animal compared to said control animal.

27. The method of claim 27 wherein the experimental animal further comprises an animal stimulated with an inflammatory stimulus.

28. A method for screening for an immunosuppressant comprising
(a) administering a test immunosuppressant to an experimental animal which is a CRP knockout animal;
(b) measuring the level of at least one cytokine which is IL-6 in said experimental animal;
(c) comparing the level of said IL-6 in said experimental animal to a control animal; and
(d) selecting an agent based on the attenuation of said IL-6 in said experimental animal compared to said control animal.

29. The method of claim 28 wherein the experimental animal further comprises an animal stimulated with an inflammatory stimulus.
